(19) United States
(12) Patent Application Publication
(10) Pub. No.: US 2012/0129963 A1
(43) Pub. Date: May 24, 2012

(54) SYNTHESIS OF NOVEL MULTIFUNCTIONAL CARDANOL'S DERIVATIVES AND THEIR USE AS HALOGEN FREE POLYURETHANIC FOAMS PRECURSORS

(76) Inventors: Elena Benedetti, Trieste (IT); Pietro Campaner, Trieste (IT); Daniele D'Amico, Tricase (IT); Andrea Minigher, Sauris (IT); Cristina Stifani, Corigliano D'Otranto (IT); Antonella Tarzia, Lecce (IT)

(21) Appl. No.: 13/261,123

(22) PCT Filed: Jul. 8, 2009
(86) PCT No.: PCT/EP2009/058686

§ 371 Date: Jan. 26, 2012

(51) Int. Cl. C08G 18/67; C07C 43/205; C07C 39/16; C08G 18/73; C08G 18/75; C08G 18/76; C08L 75/14; C07D 249/04; C07D 403/06; B82Y 30/00

(52) U.S. Cl. 521/97; 548/255; 568/630; 568/720; 521/166; 521/164; 521/177; 521/170; 521/155; 521/175; 521/82; 521/98; 977/762; 977/773; 977/902

(57) ABSTRACT

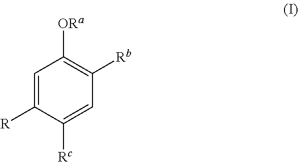

Cardanol derivative comprising one or more units of the formula and a method for obtaining such cardanol derivative.

### BACKGROUND OF THE INVENTION

The present invention relates to cardanol derivatives, their synthesis, and their use as precursors of halogen-free polyurethanic foams, as such or containing different fillers.

Cardanol represents not only a common natural product deriving from Cashew Nut Shell Liquid (CNSL) distillation, but also a useful and versatile chemical tool, because it can constitute the building blocks for novolac copolymers.

These cardanol's based novolac copolymers, but without implying any limitations thereof, can find, depending on their molecular weight, different applications such as brake linings, surface coating, foundry core oil, laminating and rubber compounding resins and adhesives, as composites and flame-retardant; the foams thus obtained can have a high density, and so being applied in building for thermal isolation, or a low density, being suitable for transportation's application (cars, trains, etc.) or filling (mattresses, shoes, etc.).

The cardanol used can be:
a mixture of meta-pentadecyl phenols, where the alkylic chain in the meta position can have 0, 1, 2 or 3 double bonds;
3-(pentadec-8-enyl)phenol (monoene cardanol), easily obtainable just properly treating cardanol by fractionated distillation, chromatography or reduction as already reported in literature;
3-pentadecylphenol (saturated cardanol), easily obtainable just adding a simple synthetic step (hydrogenation);

In particular, these starting materials lead to phenolic resin, that can be resols or novolacs, preferably novolacs, with a polymerization degree ranging but controlled, between 2 an 7, obtained by condensation of aldehydes, or their analogues, and cardanol, using suitable catalysts, where the stoichiometric ratio of the two reagents is 2-0.6/1; these products can be further modified to easily introduce other different functional groups selectively derivatizable. On the other hand, the phenoxy-urethanic foams are obtained by the reaction of the polyphenolic compounds described above and different isocyanates conveniently chosen, in presence of standard catalysts. These foams can be formulated with suitable additives for improving the physical properties of foam products, like flame retardant and anti-fume properties; in particular these polyurethanic foams contain:
Carbon nanofibers that confer flame proofing properties, eliminate foam's melt-dripping during blaze, reducing flames' propagation, and regulate cells' morphology;
Expandable graphite that increases flame resistance and reduces the evolution of combustion smokes;
Layered double hydroxides (LDH); these inorganic salts can be modified with organic molecules that can have intumescent character, flame resistance properties, reduce combustion's fumes (as already described in a recent patent, EP 1469034A1) and control cells' morphology;
Other conventional non-halogenated flame retardants, such as melamines or ammonium polyphosphates;
Benzoxazines, in order to improve the polyurethanic foam's resistance to solvents (such as tetrahydrofuran, N,N-dimethyl formamide, . . . ) and its thermal stability;
Water, pentane, etc. as foaming agents.

Cardanol is the main constituent of technical grade or distilled commercially available Cashew Nut Shell Liquid (CNSL), a side-product from the mechanical processing (hot-bath process) of the cashew nut of Anacardium occidentale, a process of importane in view of the edibility of the kernel. CNSL is a low value side-product compared with the valuable edible kernel and a widely available source of distilled cardanol and 3-m-pentadecylphenol, obtained by hydrogenation of cardanol, utilizable in fine chemical processes (Tyman, J. H. P., Chem. Soc. Rev. (1979), 8, 499-535).

Cardanol is a mixture of 3-m-pentadecylphenol, 3-(m-pentadec-8-enyl)phenol, 3-(m-pentadeca-8,11-dienyl)phenol, and 3-(m-pentadeca-8,11,14-trienyl)phenol and has been used in many industrial applications such as coatings, resins adhesives and other novel products, such as polyols that have already been partially studied as prepolymers for different industrial applications.

This structure and its derivatives may represent an useful and cheap alternative to similar molecules or prepolymers based on petroleum resource, which are however costly and scarce; cardanol and its derivatives may be used as antioxidants, and in general as stabilizers against light, air and heat, for several organic materials, e.g., flavors, foods, lubricants, polymers, and rubbers (a) Rosy, A.; Pillai, C. K. S.; Scariah, K. J., J. Appl. Polym. Sci. (1990), 41, 1765-1775; b) Menon, A. R. R.; Pillai, C. K. S.; Nando, G. B., J. Appl. Polym. Sci. (1994), 51, 2157-2164). Based on the above described properties, the synthesis of new cardanol derivatives is a straightforward key for the development of convenient industrial applications of CNSL as well as for the design of new drugs. Alkylation, condensation, nitration, halogenation and a number of other chemical transformations have all been used with different degrees of success for the selective functionalization of cardanol and its isolated components (U.S. Pat. Nos. 6,723,765; 6,583,258; 6,548,189; 6,537,636; 6,525,112; Ikeda, R.; Tanaka, H.; Uyama, H.; Kobayashi, S., Macromol. Rapid Comm. (2000), 21, 496-499; Graham, M. B.; Tyman, J. H. P., J. Am. Oil Chem. Soc. (2002), 79, 725-732; Guo, Y. C.; Menon, A. R. R.; Sonia, T. A.; Sudha, J. D., J. Appl. Polym. Sci. (2006), 102, 4801-4808; Dia, Z.; Chen, M. J., WO/2000/034219; Suwanprasop, S.; Nhujak, T.; Roengsumran, S.; Petsom, A., Ind. Eng. Chem. Res. (2004), 43, 4973-4978).

New polyphenolic cardanol's derivatives, obtained by different synthetic pathways and combinations of reagents, are both ideal for further chemical modifications (as, for example, the introduction of other different reactive groups that can be easily and selectively modified) and useful building blocks in the synthesis of halogen-free polyurethanes. These final products may be used directly as such or previously added of fillers, in order to confer different characteristics, extending their possible applications.

Polyurethane foams constitute the largest category of cellular polymeric materials; they offer an attractive balance of performance characteristics such as aging properties, mechanical strength, elastic properties, and chemical resistance, insulating properties and cost and are so produced primarily for the automotive, building, and furniture industries for use as padding, cushioning, and insulation. Depending upon production methods, urethane foams can offer different characteristics tailored for specific applications. They can make a major contribution to improving the energy efficiency of buildings when they are used as an air leakage control material or as a component of an air barrier system. They may be fastened to supporting structures (self-adhesive). They can be used for marine flotation requirements due to their good flotation properties (for example, more and more Asian shipyards are using polyurethane based elastomers in the form of a steel based sandwich plate system (SPS) in ship repairs and new buildings).

For these reasons, it is of particular interest to develop polyols or similar structures, that can be applied in the synthesis of urethanic foams, which may be easily and cheaply obtained from readily available and renewable resource material such as CNSL (and cardanol, as well), following approaches similar to the ones described for the preparation of polyols from renewable resources like vegetable oils, for which several processes are already disclosed in the literature; for example, reference may be made to U.S. Pat. No. 6,107,433 which disclosed process for preparing vegetable oil based polyol from castor oil by oxidation of the chain unsaturation using peracids and a method for making polyurethane castings using these polyols, or, once more, U.S. Pat. No. 4,825,004 a process for the production of alkane polyols starting from natural fatty acid derivatives by peracid oxidation is described. However, the use of cardanol in the preparation of polyurethane polyols is not extensively reported; in fact, considering its potential in this field, there is only a limited number of examples (EP 1930355; WO2007/077567; WO2006/003668) reported in the literature concerning the derivatization of cardanol to a multifunctional alcohol with a polyphenolic scaffold or its use as a starting material for the synthesis of polyurethanes. For example Mythili, C. V.; Malar Retna, A.; Gopalakrishnan, S., Bull. Mater. Sci., (2004), 27, 3, 235-241; Bhunia, H. P.; Jana, R. N.; Basak, A.; Lenka, S.; Nando, G. B., J. Polym. Sci. Part A: Polym. Chem. (1998), 36, 3, 391-400; Suresh, K. I.; Kishanprasad, V. S., Ind. Eng. Chem. Res. (2005), 44, 4504-4512; Mishra, D. K.; Mishra, B. K.; Lenka, S.; Nayak, P. L., Polym. Eng. Sci. (1996), 36, 8, 1047-1051; Das, T. K.; Das, D.; Bhunia, H. P.; Jana, R. N.; Basak, A.; Lenka, S.; Nando, G. B., J. Polym. Sci. Part A: Polym. Chem. (1997), 36, 3, 391-400; Nayak, R. R.; Ray, G.; Mohapatra, D. K.; Das, D.; Nayak, P. L.; Lenka, S., J. App. Pol. Sci., (1996), 70, 5, 837-842; Bhunia, H. P.; Nando, G. B.; Chaki, T. K.; Basak, A.; Lenka, S.; Nayak, P. L., Eur. Polym. J., (1999), 35, 8, 1381-1391; Das, D.; Ton That Minh Tan, J. Appl. Polym. Sci. (1996), 65, 3, 507-510; Mythili, C. V.; Malar Retna, A.; Gopalakrishnan, S., J. Appl. Polym. Sci. (2005), 98, 1, 284-288; Suresh, K. I.; Kishanprasad, V. S., Ind. Eng. Chem. Res. (2004), 44, 4504-4512; Bhunia, H. P.; Jana, R. N.; Basak, A.; Lenka, S.; Nando, G. B., J. Polym. Sci. Part A: Polym. Chem. (1997), 36 3, 391-400.

Main aim of the present invention is to develop a novel set of cardanol derivatives that allow the preparation of polyurethane-phenolic foams that have remarkable flame resistant properties and are free of halogens. Another aim of the present invention is to provide a method for the preparation of a multifunctional class of polyphenolic scaffolds based on cardanol that, in some cases, exhibit a good solubility in polar solvents including water.

It is a further object of the present invention to provide different substrates, simply applying the well-known reaction of diisocyanate and/or poly isocyanate with the hydroxyl groups of polyol co-reactants and blowing agents (such as water, dichloromethane, cyclopentane, HCFC's, . . . ), for the preparation of a wide range of polyurethanes (with a full polyurethanic character or mixed polyurethanic-polyureas or polyurethanic-polytriazoles systems).

Moreover, an aim of the present invention is to provide polyphenols that can be used as an easy to obtain, cheap and versatile starting material to be applied in the synthesis of halogen-free polyurethanic foams, both flexible and rigid, simply choosing the suitable structure of the starting polyol, with one or more phenolic unit condensed or variably functionalized.

Further details and advantages will be disclosed in a preferred, but not exclusive, way of execution of the object of the present invention.

### SUMMARY OF THE INVENTION

An aspect of the present invention refers to cardanol derivatives comprising one or more units of the formula

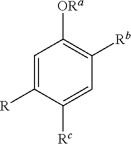

wherein

R is

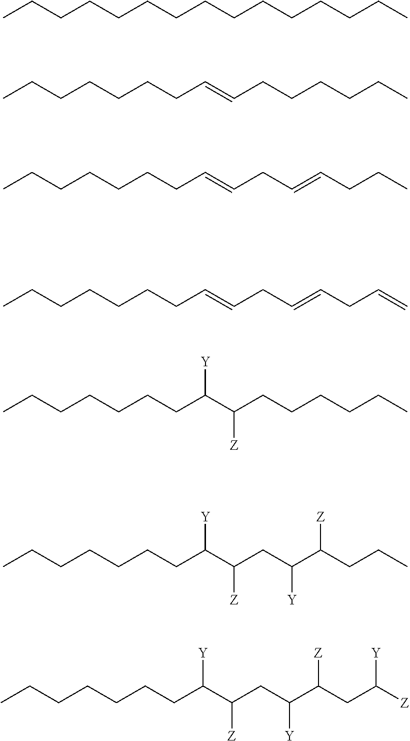

Y is H, OH, NH₂, N₃,

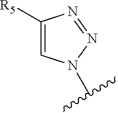

Z is H, OH, NH₂, N₃,

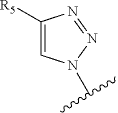

R^{a} is H or —CH₂—CHR₃)ₙ—(CH₂—R₂

R^{b} is a bond or —(CHR₃)ₙ—(CH₂)ₘ—(CHR₄)ₚ

R₁ is H, OH, NH₂

R₂ is H, OH, NH₂, N₃, triazole, N(CH₂CH₂OH), N(CHCH₃—CH₂OH), OCH₂CH(OH)CH₂OH

R₃, R₄ and R₅ are independently H, alkyl, Ar, phenyl, optionally substituted

n is 0, 1, 2, 3, 4

m is 0, 1, 2, 3, 4

p is 0, 1, 2, 3, 4

and R^{c} is H or —N—(CH₂—CH₂OH)₂ or —N—(CHCH₃—CH₂OH)

In another aspect the present invention refers to a method for obtaining a cardanol derivative according to the present invention comprising the steps of:

(a) providing a cardanol selected from saturated cardanol, cardanol monoene, cardanol diene, cardanol triene or a mixture thereof;

(b) condensing said cardanol or cardanol mixture optionally with an aldehyde or acetal or a second phenol and aldehyde or acetal.

In another aspect the present invention refers to a method for obtaining a polyurethane comprising the steps of:

(a) providing a cardanol derivative above described;

(b) reacting said polyolic compounds with polyisocyanates in the presence of catalysts, if required;

(c) addition of blowing agents.

In another aspect the present invention refers to a method for preparing a polyurethane comprising the steps of:

(a) providing cardanol derivatives above described or obtained with a method of the present invention;

(b) reacting said cardanol derivatives with isocyanates in the presence of catalysts, if required, and addition of a blowing agent to produce polyurethane.

### DESCRIPTION OF THE PREFERRED EMBODIMENTS

The present invention refers to cardanol derivatives comprising one or more units of the formula

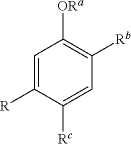

wherein

R is

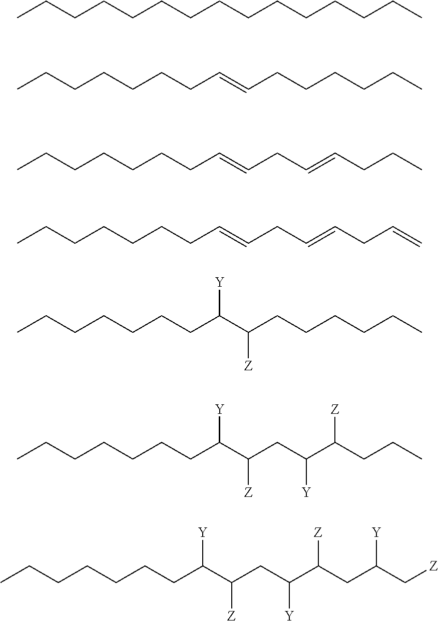

Y is H, OH, NH₂, N₃,

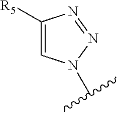

Z is H, OH, NH₂, N₃,

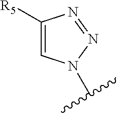

R^{a} is H or —CH₂—CHR₂—CH₂-R₂

R₁ is H, OH, NH₂

R₂ is H, OH, NH₂, N₃, triazole, N(CH₂CH₂OH), N(CHCH₃—CH₂OH), OCH₂CH(OH)CH₂OH

R^{b} is a bond or —(CHR₃)ₙ—(CH₂)ₘ—(CHR₄)ₚ

R₃, R₄ and R₅ are independently H, alkyl, Ar, phenyl, optionally substituted

n is 0, 1, 2, 3, 4

m is 0, 1, 2, 3, 4

p is 0, 1, 2, 3, 4

and R^{c} is H or —N—(CH₂—CH₂OH)₂ or —N—(CHCH₃—CH₂OH)

In a preferred embodiment of the invention the cardanol derivative is selected from the group consisting of:

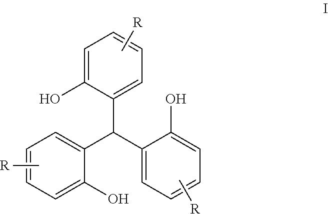

wherein R is

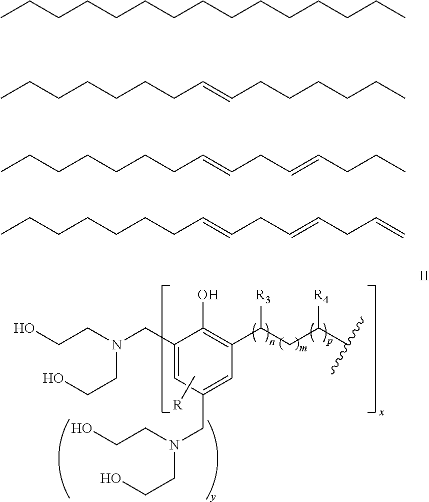

wherein x is 1, 2, 3, 4, 5, or 6;

y is 0 or 1;

R is

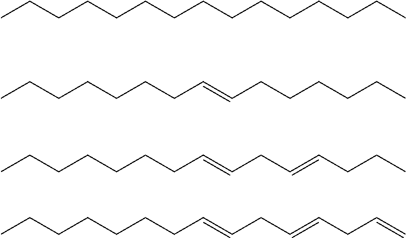

and R₃, R₄, n, m, p are as defined above;

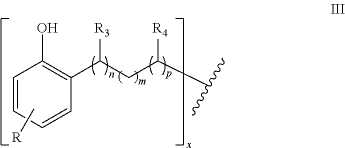

wherein x is 1, 2, 3, 4, 5, or 6;

R is

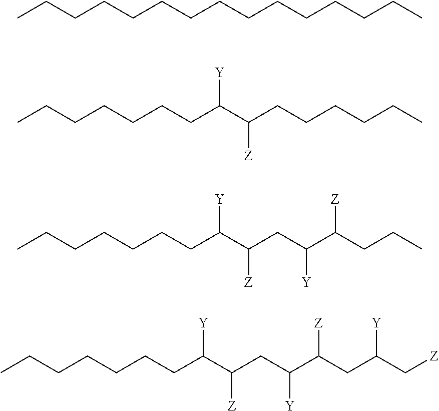

and R₃, R₄, n, m, p are as defined above;

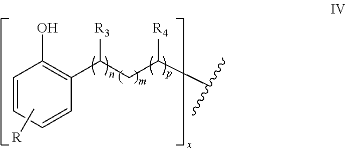

wherein x is 1, 2, 3, 4, 5, or 6;

R is

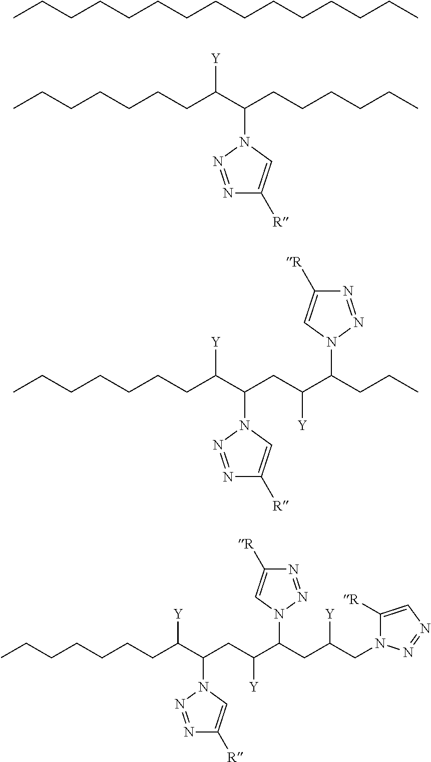

and R₃, R₄, n, m, p are as defined above;

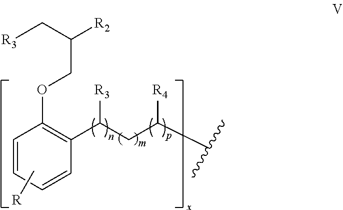

wherein x is 2, 3, 4, 5, or 6;

R is

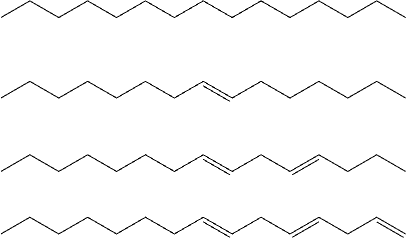

and R₂, R₃, R₄, n, m, p are as defined above;

and

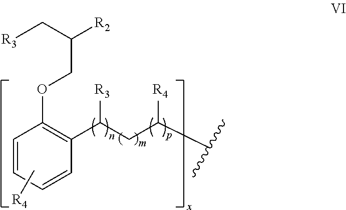

wherein x is 2, 3, 4, 5 or 6;

R is

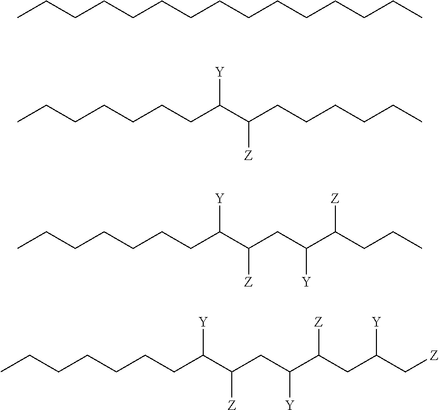

and R₂, R₃, R₄, n, m, p are as defined above.

The above-mentioned structures are defined as follows:

| Structure | Brute Formula |
|---|---|
| 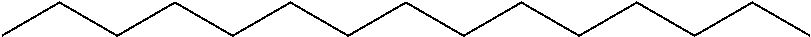 | —CH₂—(CH₂)₁₃—CH₃ |
| 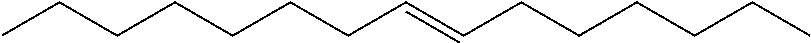 | —CH₂—(CH₂)₆—CH═CH—(CH₂)₅—CH₃ |
| 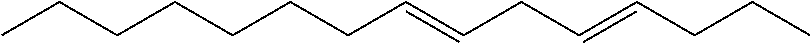 | —CH₂—(CH₂)₆—CH═CH—CH₂—CH═CH—(CH₂)₂—CH₃ |
| 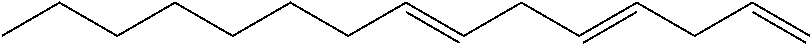 | —CH₂—(CH₂)₆—CH═CH—CH₂—CH═CH—CH₂—CH═CH₂ |
| 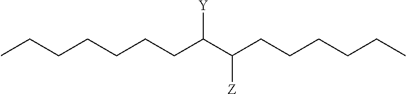 | —CH₂—(CH₂)₆—CHY—CHZ—(CH₂)₅—CH₃ |
| 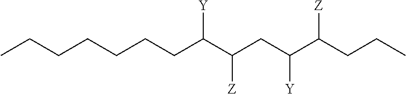 | —CH₂—(CH₂)₆—CHY—CHZ—CH₂—CHY—CHZ—(CH₂)₂—CH₃ |
| 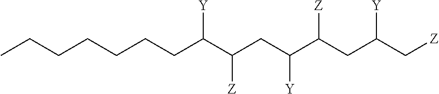 | —CH₂—(CH₂)₆—CHY—CHZ—CH₂—CHY—CHZ—CH₂—CHY—CHZ |
| 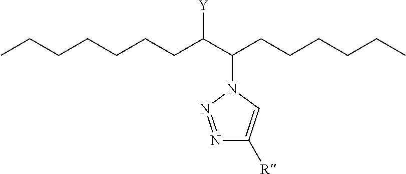 | 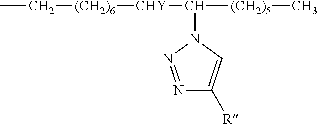 |
| 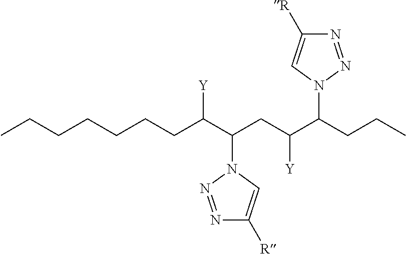 | 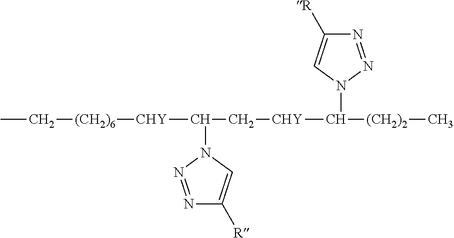 |
| 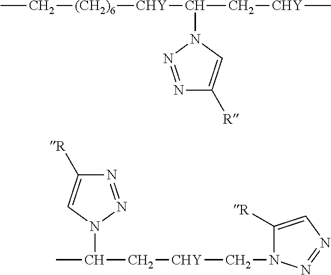 | |

In other words, cardanol derivatives of the present invention, branched or not, are characterized by the presence of a polyphenolic scaffold with 2 as the minimum degree of polymerization (dimeric structures), with a variable, but well determine number of OH groups (including the phenolic ones and other subsequently introduced on the side chains), with a variable, but well determined number of NH₂ and N₃ groups as well, which can be successfully used in the preparation of polyurethanes, polycarbonates, polyoxiranes, polyols, polytriazoles, polyaminoalcohols, or any their combination thereof.

Another aspect of the present invention refers to provide different substrates, simply applying the well-known reaction of diisocyanate and/or poly isocyanate with the hydroxyl groups of polyol co-reactants and blowing agents (such as water, dichloromethane, cyclopentane, HCFC's, etc.), for the preparation of a wide range of polyurethanes (with a full polyurethanic character or mixed polyurethanic-polyureas or polyurethanic-polytriazoles systems). The polyurea polymer polyols, for example, may be used in the manufacture of flexible polyurethane foams which are firmer and stronger than similar products using conventional polyols (see for example, U.S. Pat. No. 4,296,213); or, furthermore, structures characterized by the presence of triazolic units may present good electrical properties (Martwiset, S.; Woudenberg, R. C.; Granados-Focil, S.; Yavuzcetin, O.; Tuominen, M. T.; Coghlin, E. B., Solid State Ionics (2007), 178, 1398-1403). The new polyurethanes obtainable can also be added with different fillers (e.g. carbon nano-fibers, multi-layered nano-graphite, expandable graphite, graphite oxide, phyllosilicate), in order to increase mechanical (see for example WO2006073712), flame resistant or fire retardant properties (WO2006033981), resistance to the solvents and thermal stability (Takeichi, T.; Guo, Y.; Agag, T. J. Polym. Sci. Part A: Polym. Chem. (2000), 38, 22, 4165-4176).

Moreover, another aspect of the present invention regards to a method for obtaining a cardanol derivative described above comprising the step of:

(a) providing a cardanol selected from saturated cardanol, cardanol monoene, cardanol diene, cardanol triene or a mixture thereof;

(b) condensing of said cardanol or cardanol mixture optionally with an aldehyde or acetal or a second phenol and aldehyde or acetal.

The cardanol can be freshly distilled before the condensing step, and characterized by chromatography, purified and hydrogenated to obtain a saturated cardanol and/or a cardanol monoene.

Preferably, the aldehyde is selected from the group consisting of alkylic aldehydes and acrylic aldehydes.

The condensation can be carried out in the presence of a halogenated solvent and a Lewis catalyst, thereby a polyol of formula I is obtained.

The condensation can be carried out with paraformaldehyde in the presence of diethanolamine, thereby a polyol of formula II is obtained. The condensation can also be carried out with a second phenol and an aldehyde or acetal in the presence of an acidic catalyst to obtain a polyol having a polyphenolic structure.

The said second phenol can be cardanol and/or bear one or more substituents, or is a polyol obtained with the above-described method.

Preferably, the aid substituents are selected from the group consisting of phenyl, alkyl, alkenyl, aryl, amino, halogen, and hydroxy.

Said acid catalyst can be selected from the group consisting of mineral, organic and Lewis acids.

The method of the present invention can further comprise epoxidation of said polyol having a polyphenolic structure with an epoxidizing agent to obtain an epoxidized product containing oxiranic rings, followed by nucleophilic oxiranic ring opening with a nucleophilic agent.

Preferably, said nucleophilic agent is selected from the group consisting of hydrogen, alcohols, ammonia, azides, amines.

Preferably, the said epoxidation agent is selected from the group consisting of hydrogen peroxide, epichlorhydrin, peroxyformic acid, peroxyacetic acid, trifluoroperoxyacetic acid, benzyloxyperoxy formic acid, m-chloroperoxybenzoic acid, and combinations thereof.

Preferably, the said epoxidizing agent is a peracid, whereby obtaining a polyol of formula III.

Preferably, the said nucleophilic agent of the present invention is an azide, and the method further comprises a 1,3-dipolar cycloaddition reaction with an alkyne in the presence of a suitable catalysts, preferably copper metal or copper sulfate with sodium ascorbate, wherein copper(I) is in the catalytic species, thereby obtaining a polyol of formula IV.

The method of the present invention can further comprise a functionalization of the phenolic OH groups with epichlorohydrin thereby obtaining a functionalized product containing oxiranic rings, followed by nucleophilic oxiranic ring opening with a nucleophilic agent, whereby obtaining a polyol of formula V.

Preferably, the method further comprises a functionalization of the phenolic OH groups with epichlorohydrin and epoxidation of the polyphenolic structure with peracids to obtain an epoxidation product containing oxiranic rings, nucleophilic oxiranic rings opening with an azide and a 1,3-dipolar cycloaddition reaction with an alkyne in the pr4esence of a suitable catalyst, preferably copper metal or copper sulfate with sodium ascorbate, wherein copper (I) is in the catalytic species, thereby obtaining a polyol of formula VI. Preferably, said alkyne bears substituents are selected from the group consisting of acetylene, propyne, phenylacetylene, but-1-yne, but-2-yne. Another aspect of the present invention refers to a method for obtaining a polyurethane comprising the steps of:

(a) providing a cardanol derivative described above;

(b) reacting said polyolic compounds with polyisocyanates in the presence of catalysts, if required;

(c) addition of blowing agents.

Another aspect of the present invention refers to a method for preparing a polyurethane comprising the steps of:

(a) providing cardanol derivatives of the present invention or obtained with a method above described;

(b) reacting said cardanol derivatives with isocyanates in the presence of catalysts, if required, and addition of a blowing agent to produce a polyurethane.

Preferably, the isocyanates have at least a NCO reacting group selected from the group consisting of 1,4-diidocyanatobutane; 1,6-diisocyanatohexane, 1,5-diisocyanato-2,2-dimethylpentane, 2,2,4- and 2,4,4-trimethyl-1,6-diisocyanatohexane, 1-isocyanate-3,3,5-trimethyl-5-isocyanatomethylcyclohexane, 1-isocyanato-1-methyl-4-(3)-isocyanato methylcyclohexane, bis-(4-isocyanatocyclohexyl)methane, 1,10-diisocyanatodecane, 1,12-diisocyantododecane, cyclohexane 1,3-and 1,4-diisocyanate, xylylene diisocyanate isomers, triisocyanatononane, 2,4-diisocyantotoluene or its mixture with 2,6-diisocyanatotoluene, diisocyanatodiphenylmethane or technical polyisocyanate mixtures of the diphenylmethane series, or a mixture thereof Preferably, the amounts of isocyanate and polyphenolic scaffolds are chosen so as to given a NCO:OH equivalent ratio of from 0.5:1 to 2.0:1, preferably from 0.8:1 to 1.5:1.

Said cardanol derivatives can be used in mixture with other polyols selected from the group consisting of glycerol, sugars, canola oil deriving polyols, soybean oil based polyols, linseed oil based polyols, and castor oil based polyol, in a weight ratio from 95:5 to 5:95.

Preferably, the catalyst is selected from the group consisting of tertiary amines, metal salts, or mixture thereof Preferably, the tertiary amines are selected from the group consisting of triethylamine, pyridine, methylpyridine, benzylmethylamine, N,N-endoethylenepiperazine, N-methylpiperidine, pentamethyldiethylenetriamine, N-,N-dimethylaminocyclohexane, N,N′-dimethylpiperazine.

Preferably, the metal salts are selected from the group consisting of iron(III) chloride, zinc chloride, zinc 2-ethylcaproate, tin(III)octoate, tin(II)ethylcaproate, tin(II)palmitate, dibutyltin(II)dilaurate, molybdenum glycolate, and/or a mixture thereof.

Preferably, the blowing agents are selected from the group consisting of water, carbon dioxide, fluorocarbons, chlorofluorocarbons, hydrofluorocarbons, perfluorocarbons, and low boiling hydrocarbons. Said method can further comprise a surfactants addition, said surfactants selected from the group consisting of silicones, fluoro based surfactants, or organic based surfactants.

In a preferred embodiment of the invention, the polyurethanes are added of additives selected from the group consisting of surface-active substances, internal release agents, fillers, dyes, pigments, flame retardants, hydrolysis preventives, microbiocides, leveling assistants, antioxidants, carbon nano-fibers, nano-graphite, expandable graphite, graphite fine powder, graphite oxide, benzoxazines, phyllosilicate, and/or a mixture thereof.

In other words, the present invention describes the development of different methods for the synthesis of multifunctional alcohols or branched alcohols or amino alcohols or azido-alcohols or triazolic-alcohols, or their combinations thereof, of the general formulas given above, comprising the step of cardanol's hydrogenation under standard catalytic conditions and 3-(pentadec-8-enyl)phenol (cardanol monoene) isolation, using synthetic conditions already reported (such as fractional distillation, as described in Bhunia, H. P.; Nando, G. B.; Basak, A.; Lenka, S.; Nayak, P. L., Eur. Polym. J. (1999), 35, 9, 1713-1722); argentation liquid chromatography, as described in Mythili, C. V.; Malra Retna, A.; Gopalakrishnan, S. Bull. Mater. Sci. (2004), 27, 3, 235-241; reduction, as described in Tyman, J. H. P.; Johnson, R. A., J. Amer. Oil Chem. Soc. (2007), 84, 573-578).

In particular, hydrogenation has been performed under novel conditions, at room temperature and ambient pressure, using ammonium formate and Pd/C as the catalytic system, carrying on the reaction with or without any solvent. Monomeric cardanol (as a mixture of the four possible isomers, as saturated cardanol or as cardanol monoene) was subjected, for example, to a condensation reaction with diethanolamine and paraformaldehyde in suitable ratios (preferably cardanol:diethanolamine:paraformaldehyde 1:1:1 or 1:2:2) or modified by acid or basic hydrolysis, thus obtaining some functionalized monomers, that can be used as such or more preferably further polymerized, as described as follows.

The polyphenolic scaffolds, both the linear and the branched ones, described in the present patent are obtained mixing cardanol (hydrogenated or not, eventually previously functionalized as described above), a second phenol (a different one or cardanol, hydrogenated or not, as well, eventually previously functionalized as described above), an acidic catalyst conveniently chosen and an aldehyde (or its analogues).

These structures can be used as such, or can be further derivatized in order to introduce other functional groups; for example, the novolacs thus obtained can be efficiently epoxidized using a peracid (or simply hydrogen peroxide) and then catalitically hydrogenated (or eventually reduced using other standard reducing agents, such as lithium aluminum hydride) to introduce other hydroxy groups on the flanking chains. The same epoxy groups can be efficiently hydrolyzed with ammonia, giving amino-alcohols or, otherwise, nucleophilically opened with sodium azide, giving azido-alcohols. These last structures can be successfully used in the synthesis of triazolic-polyphenolic cardanol's derivatives, using the standard conditions needed by 1,3-dipolar cycloaddition (Huisgen cycloaddition).

These kinds of approach and chemical modifications have been successfully used in the synthesis of other cardanol's highly functionalized derivatives, starting from novolacs with an oxirane ring on each phenolic unit; in this case, the flanking alkylic chain is not altered during the synthesis and so being useful to decrease structures' stiffness. Last class of structures object of this invention joins together the characteristics of the ones previously described, combining the derivatization on the aromatic hydroxy group with the modifications of the long alkyl chain.

The main finding of the present invention is the observation that new different and multifunctional cardanol's derivatives are obtained only with a minimum change in the reaction's conditions, affording a library of structures easy and ready to use, with a hydroxy number between 180 and 600, with an average functionality between 2.5 and 5.

Novolac resins are formed by acid or metal ion catalyzed co-condensation of phenols with formaldehyde, its derivatives or other suitable aldehydes.

At the beginning, considering that the starting cardanol is a raw material of natural origin, and so characterized by a wide variability in terms of composition, in order to choose the best experimental conditions, as the first step the percentage of each component was determined by High Performance Liquid Chromatography (HPLC), using a reverse phase column and eluting the sample isocratically, with a 85/15 methanol/water mixture (this technique has extensively, together with NMR, been used also in the characterization of cardanol's derivatives). On the basis of the data collected, and in particular on the degree of purity determined, cardanol has eventually been purified carrying out a distillation under reduced pressure and collecting the starting material, at 215-220° C. at 4-5 mmHg, as a yellow oil that has been immediately used or stocked avoiding exposure to light or added of suitable anti-oxidants (e.g. hydroquinone). Once obtained, this phenol, eventually hydrogenated or conveniently treated to give the monoene (and so giving a final homogenous product, considering the nature of the flanking residues), has been used as such or for the synthesis of different kinds of novolacs, to be used as precursors of some of the polyols herein described, or differently functionalized monomers; in the first case, for example, cardanol is reacted with itself in the presence of chloroform, zinc chloride as a catalyst under reflux, giving a trimer (following an approach similar to the one described by Driver, J. E.; Lai, T. F., J. Chem. Soc. (1958), 3009-3015). On the other hand, if cardanol is reacted with a suitable aldehyde (and eventually with a different phenol to give mixed novolacs) in the presence of an acidic catalyst (trifluoracetic acid, oxalic acid, formic acid, etc.) a wider range of structures can easily be recovered. For example, reacting cardanol with oxalic acid and formalin under reflux conditions a linear novolac is recovered, whose molecular weight and physical properties, as in all the other examples here described, depend on the cardanol/formaldehyde ratio. In order to reduce the possible disadvantages deriving from the fact that formaldehyde is not fully used up, contains a high amount of water and the effluent may contain unreacted formaldehyde which causes environmental pollution, different synthetic strategies have been investigated, here described and patented; for example, following approaches similar to the ones already described in the literature (a) Sun, H.-B.; Hua, R.; Yin, Y., Tetrahedron Lett. (2006), 47, 14, 2291-2294; b) Banihashemi, A.; Rahmatpour, A., J. Chem. Res. Synop. (1999), 6, 390-391) we have developed different experimental conditions based on the use of solid formaldehyde analogues (acetals) such as 1,3,5-trioxane and the less expensive paraformaldehyde, that, under room temperature or reflux conditions depending on the acidic catalyst chosen, lead to highly pure linear novolacs. The same approach has been successfully used in the synthesis of branched, monodisperse cardanol's based novolac, using di-acetals as the aldehyde source. All these approaches have been used both on hydrogenated cardanol, monene cardanol and mixture of cardanol's four isomers, giving novolacs with different viscosity and colour (from yellow to amber to brown).

The products, both the ones described above and the others cited later, once purified by common chromatography techniques or by distillation under reduced pressure, have all been characterized both by NMR spectroscopy and MS spectrometry, and by titration techniques, determining the average number of chemical functionalities obtained or introduced in the different synthetic steps (for example, the number of phenolic OH groups have been titrated using methyl orange as an indicator and a normalized KOH solution or by acetylation, as described in ASTM E222-00(2005)e1; the hydroxyl value of the different polyols obtained has been determined by the pyridine-acetic anhydride method, as described in the Annual Book of ASTM Standards, ASTM International: West Conshohocken, Pa., 1983, 6.03; the number of double bonds present on the prepolymers has been determined by common protocols, e.g. by iodine number or bromination, like the ones described in Anal. Chem. (1968), 40, 1, 134-139, while the number of epoxide groups has been measured following what suggested ASTM D1652-97). The novolacs containing double bonds on the side chains have been epoxidized, using common peracids or hydrogen peroxide, and used in the following steps. Hydrolyzing the oxirane rings with hydrogen at room temperature and atmospheric pressure (or with standard reducing agents) polyols with an OH group for each epoxy are recovered; if the hydrolysis is performed with concentrated ammonia polyaminoalcohols are obtained, with an OH and a NH₂ group from each epoxy group; if the hydrolysis involves an azide, the final product, with an OH and a N₃ group deriving from each epoxy ring, is a polyazidoalcohol that can easily further react, giving a polytriazolic alcohol, with an alkyne in the presence of a Cu/Cu(I) system; if the nucleophilic oxiranic ring opening is performed with diethanolamine, for example, 3 OH groups are obtained for each epoxy group, two deriving from diethanolamine itself and one form the epoxy group.

A similar approach involves the modification of the OH phenolic groups, that can be modified with epichlorhydrin using standard conditions, introducing oxirane rings in a position different from the one described above; these new functionalities can react in the same conditions already described. Combining the two approaches, new chemical entities have been introduced both on the side chains and the phenolic rings, increasing the number of reactive functionalities.

Furthermore, the novolacs as such (both the ones deriving from saturated cardanol, monoene or from the mixture of the four isomers), can be reacted, as previously described for monomeric cardanol, with diethanolamine, in the presence of paraformaldehyde.

These new cardanol based products have been reacted with suitable commercially available isocyanates in the presence of standard catalysts, with or without additives like the ones cited above, giving few different polyurethanes and so confirming the value of the structures synthesized.

Experimental details are given in the following examples, which are provided by way of illustration only and should not be construed to limit the scope of the invention.

### EXAMPLES

### Example 1

Freshly distilled cardanol (5 g, 0.016 mol), as a mixture of its four components (saturated, mono-,di-, triene), is placed in a three-necked round bottom flask, dissolved in methanol (25 mL) and added of ammonium formate (12.6 g, 0.2 mol); vacuum is then applied to the system in order to remove any traces of air and then flushed with nitrogen for a couple of minutes. Pd/C 10% wet (10% by weight with respect to the substrate) is then added under nitrogen atmosphere and the system is left under stirring at room temperature for 4 h; the catalyst is removed by filtration through a short pad of Celite, the residue is diluted with dichloromethane, washed with water, brine, dried over anhydrous sodium sulfate. Distillation of the solvent under reduced pressure affords 4.86 g of a solid that can be used without any further purification.

### Example 2

Hydrogenated cardanol (2 g, 6.57 mmol) is added of chloroform (175 L, 2.19 mmol) and ZnCl₂ (3.07 mmol) and left at 130° C. for 7 h. The residue is then left cooling at room temperature, diluted with ethyl acetate, extracted with 5% (w/v) aqueous NaOH, acidified with acetic acid, washed with water, brine and dried over anhydrous sodium sulfate. The solvent is then removed under reduced pressure, the residue purified by flash chromatography on a silica gel column using a petroleum ether/diethyl ether mixture as eluant (gradient from 7/3 to 1/1).

### Example 3

Hydrogenated cardanol (2 g, 6.57 mmol) is dissolved in a 4/6 mixture trifluoroacetic acetic/chloroform (25 mL) and added of 1,3,5-trioxane (2.19 mmol); the mixture is then left stirring for 6 h at room temperature. The solvent is then removed under reduced pressure, the residue is subsequently co-evaporated with diethyl ether and finally purified by flash chromatography on a silica gel column, using a petroleum ether/diethyl ether mixture as eluant (gradient from 7/3 to 1/1).

### Example 4

Hydrogenated cardanol (1 g, 3.28 mmol) is dissolved in a 4/6 mixture trifluoroacetic acetic/chloroform (15 mL) and added of paraformaldehyde (4.92 mmol); the mixture is then left stirring for 25 min at room temperature. The solvent is then removed under reduced pressure, the residue is subsequently co-evaporated with diethyl ether and a high crystalline solid is recovered.

### Example 5

Cardanol (10 g, 0.032 mol), as a mixture of its four components (saturated, mono-,di-, triene), is dissolved in toluene and heated at 70° C. in a three-necked round bottom flask, then added of a pre-incubated (at 70° C. for 15 min) solution of 1,3,5-trioxane (590 mg, 3.94 mmol) and oxalic acid (2.48 mg, 3.94 mmol) in toluene. The reaction is then carried out in a Dean-Stark apparatus, measuring the amount of water produced; once the reaction is over, the solution is washed with water, brine and dried over anhydrous sodium sulfate. The solvent is then removed under reduced pressure and the crude product purified by flash chromatography on a silica gel column, using a petroleum ether/diethyl ether mixture as eluant (gradient from 7/3 to 1/1) or chloroform/diethyl ether 9/1; the crude novolac can also be purified by distillation under reduced pressure, removing the unreacted cardanol (at 220° C. and 4 mmHg).

### Example 6

Cardanol (10 g, 0.032 mol), as a mixture of its four components (saturated, mono-,di-, triene), is placed in a three-necked round bottom flask, heated at 70° C. and then added of a pre-incubated (for 15 min at rt) solution of a 37% aqueous formaldehyde solution (590 mg, 3.94 mmol) and oxalic acid (248 mg, 3.94 mmol). The reaction is then carried out at 100° C. for 9 h, distilling water away; once the reaction is over, the solution is diluted with chloroform, washed with a NaHCO₃ saturated aqueous solution, water, brine and dried over anhydrous sodium sulfate. The solvent is then removed under reduced pressure and the crude product purified by flash chromatography on a silica gel column, using a petroleum ether/diethyl ether mixture as eluant (gradient from 7/3 to 1/1) or chloroform/diethyl ether 9/1; the crude novolac can also be purified by distillation under reduced pressure, removing the unreacted cardanol (at 220° C. and 4 mmHg).

### Example 7

Cardanol (300 g, 1 mol), as a mixture of its four components (saturated, mono-,di-, triene), is placed, in the presence of formic acid (24.6 mL, 10% w/w with respect to cardanol) in a three-necked round bottom flask, heated at 70° C. and then added of paraformaldehyde (18.05 g, 0.6 mol). The reaction is then carried out at 100° C. for 8 h; once the reaction is over, the crude product is distilled, removing water and the acidic catalyst first and then, just increasing the temperature and the vacuum, the unreacted cardanol, with a final 75% yield of pure novolac.

### Example 8

Hydrogenated cardanol (2 g, 6.57 mmol) is dissolved in a 4/6 mixture trifluoroacetic acetic/chloroform (25 mL) and added of 2,5-dimethoxytetrahydrofuran (2.19 mmol); the mixture is then left stirring for 6 h at room temperature. The solvent is then removed under reduced pressure, the residue is subsequently co-evaporated with diethyl ether and finally purified by flash chromatography on a silica gel column, using a petroleum ether/diethyl ether mixture as eluant (gradient from 7/3 to 1/1) or chloroform/diethyl ether 9/1.

### Example 9

Freshly distilled cardanol (5 g, 0.017 mol), as a mixture of its four components (saturated, mono-,di-, triene) and phenol (1.57 g, 0.017 mol) are placed, in the presence of formic acid (1.75 mL, 10% w/w with respect to the phenols) in a three-necked round bottom flask, heated at 70° C. and then added of paraformaldehyde (301 mg, 0.6 mol). The reaction is then carried out at 100° C. for 8 h; once the reaction is over, the crude product is distilled, removing water and the acidic catalyst first and then, just increasing the temperature and the vacuum, the unreacted phenols, with a final 71% yield of pure mixed novolac.

### Example 10

A solution of m-chloro-perbenzoic acid (mCPBA) (1.2 eq) in dichloromethane is added to a solution of novolac (1 eq) in dichloromethane, cooled to 0° C. The mixture is then left stirring at room temperature for 16 h, then diluted with dichloromethane, washed with a 10% (w/v) sodium metabisulphite aqueous solution, a NaHCO₃ saturated aqueous solution, brine and finally dried over anhydrous sodium sulfate. The solvent is then removed under reduced pressure and the residue purified by flash chromatography on a silica gel column, using a dichloromethane/methanol mixture as eluant (gradient from 8/2 to 1/1).

### Example 11

In a 100 mL, three-necked, round-bottomed flask fitted with a mechanical overhead stirrer, addition funnel, and thermometer are placed 2 g (3.3 mmol) of cardanol (as a mixture of its four components), 15 mL of methanol, 1.88 mL (0.018 mol) of acetonitrile, and 170 mg (1.69 mmol) of potassium bicarbonate. To the resulting heterogeneous mixture is added dropwise 2.66 g (0.011 mol) of 30% hydrogen peroxide with cooling at a rate that maintains the temperature of the reaction at 25-35° C. Following the addition of hydrogen peroxide, the ice bath is removed and the reaction mixture is allowed to stir at room temperature overnight. The reaction mixture is diluted with brine, then extracted three times with dichloromethane, the combined organic phases are dried over sodium sulfate and the solvent removed under reduced pressure by rotary evaporation.

### Example 12

The epoxidized novolac (1 eq) is dissolved in methanol, added of Pd/C 10% and left stirring under hydrogen atmosphere for 16 h at room temperature. The catalyst is then filtered off through Celite; distillation of the solvent under reduced pressure affords a polyol that doesn't need any further purification.

### Example 13

The epoxidized novolac (1 eq) is dissolved in isopropanol, added of concentrated ammonia and left stirring for 8 h at 80° C. The solvent is removed under reduced pressure, affording a polyaminoalcohol that doesn't need any further purification.

### Example 14

NaN₃ (2.61 mmol), NH₄Cl (1.68 mmol) and an epoxidized novolac (0.52 mmol) in 6 mL of 8:1 MeOH—H₂O were heated at 80° C. in a sealed tube for 8 h. The reaction mixture was cooled to room temperature and partitioned between diethyl ether and 10% aqueous NaHCO₃. The aqueous phase was extracted with diethyl ether, and the combined organic phases were washed with brine and dried over sodium sulfate. The solvent was rotary evaporated and the resulting crude azide was used without further purification.

### Example 15

A generic polyphenolic polyazidoalcohol (1 eq), obtained using one of the protocols described above, is dissolved in tBuOH (or DMSO) and added of phenylacetylene (1 eq) and a 300 μM CuSO₄ aqueous solution in the presence of Cu shavings. The system is stirred for 16 h at 50° C., added of few drops of concentrated ammonia, diluted with dichloromethane, washed with water, brine and dried over Na₂SO₄. Distillation of the solvent under reduced pressure afforded a polytriazolic polyphenolic scaffold that can be used without any further purification (94%).

### Example 16

In the first step a generic novolac was reacted with epichlorohydrin under alkaline conditions to give the monoglycidyl ether. In a typical experiment, novolac (1 eq) in a 500-mL round-bottom flask fitted with a mechanical stirrer, thermometer, and dropping funnel was heated to 95° C. Then 0.1% of anhydrous ZnCl₂ was added. The required quantity of epichlorohydrin (1.6 eq) was then added dropwise while the temperature was maintained. After its addition the reaction was continued for 2-3 h. Then a stoichiometric amount of sodium hydroxide (1.6 eq in 100 mL water) was added dropwise. The reaction temperature was increased to 100° C. and heating continued for 2-3 h. The product was separated and washed with excess water to remove the by-product sodium chloride. It was then dried over anhydrous Na₂SO₄ (83%).

The epoxy-novolac thus obtained (1 eq) was mixed with twice its weight of 10% H₂SO₄ in a 250-mL three-neck round-bottom flask, fitted with a mechanical stirrer, thermometer, and reflux condenser. The reaction mixture was heated under reflux for about 10 h. The product, extracted in ether, was washed with water until neutral to litmus and dried over anhydrous Na₂SO₄ (95%).

### Example 17

An epoxy-novolac (1 eq) obtained following the approach described in Example 16 and diethanol amine (1.2 eq) were reacted at reflux in the presence of ethanol in a 250-mL round-bottom flask, fitted with a mechanical stirrer, thermometer, and a reflux condenser. After 7 h ethanol was removed from the product on a rotary evaporator. The product was separated and washed with a water-ethanol mixture (1:1) and finally with water to remove excess diethanolamine, if any. It was dried over anhydrous Na₂SO₄ (92%).

### Example 18

Monomeric cardanol (1 eq) is heated at 100° C., under mechanical stirring, in the presence of diethanolamine (1 eq) and paraformaldehyde (1 eq), measuring the amount of condensation water produced during the reaction using a Dean-Stark apparatus. Once the reaction is complete, the system is cooled at room temperature, dried over anhydrous Na₂SO₄ to remove traces of water, affording a crude product that doesn't need any further purification.

### Example 19

A typical procedure for the synthesis of a rigid polyurethanic foam comprises the step of mixing the polyol and the catalyst (e.g. DBTDL), if, for example there are not any tertiary amino groups in the polyol; the diisocyanate (PMDI) is then added dropwise, eventually using a suitable blowing agent.

1-30. (canceled)

31. A cardanol derivative comprising one or more units of the formula 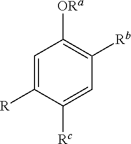
wherein
R is 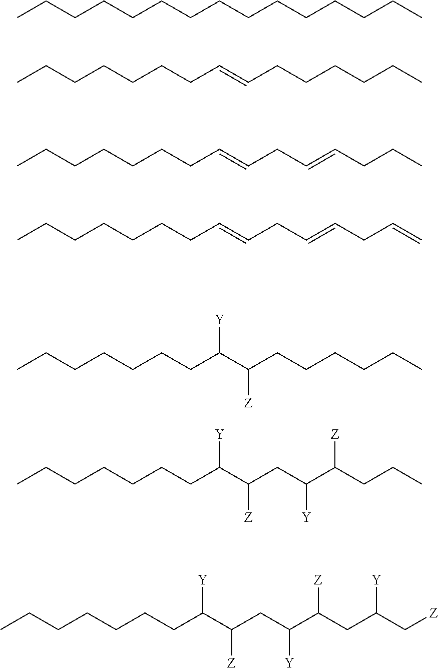
Y is H, OH, NH₂, N₃, 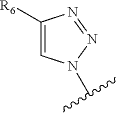
Z is H, OH, NH₂, N₃, 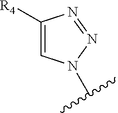
R^{a} is H or —CH₂—CHR₁—CH₂—R₂
R₁ is H, OH, NH₂
R₂ is H, OH, NH₂, N₃, triazole, N(CH₂CH₂OH), N(CHCH₃—CH₂OH), OCH₂CH(OH)CH₂OH
R^{b} is a bond or —(CHR₃)ₙ—(CH₂)ₘ—(CHR₄)ₚ
R₃, R₄ and R₅ are independently H, alkyl, Ar, phenyl, optionally substituted
n is 0, 1, 2, 3, 4
m is 0, 1, 2, 3, 4
p is 0, 1, 2, 3, 4
and R^{c} is H or —N—(CH₂—CH₂OH)₂ or —N—(CHCH₃—CH₂OH),
provided that when R is 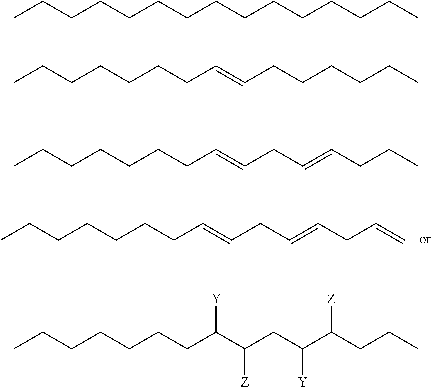
at least one of R^{a}, R^{b} or Re is not H.

32. The cardanol derivative according to claim 31, selected from the group consisting of: 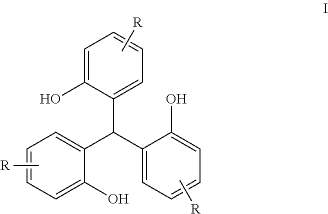
wherein R is 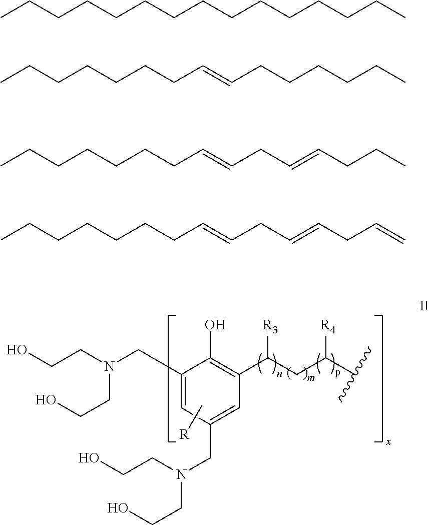
wherein
x is 1, 2, 3, 4, 5, or 6;
R is 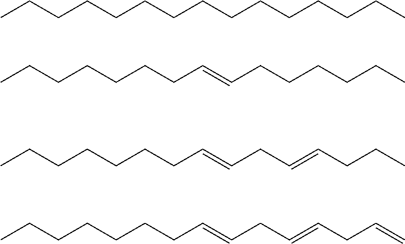
and R₃, R₄, m, n, p are as defined in claim 31; 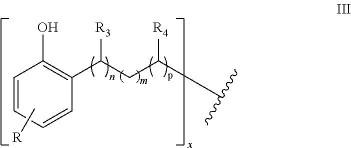
wherein
x is 1,2, 3,4, 5, or 6;
R is 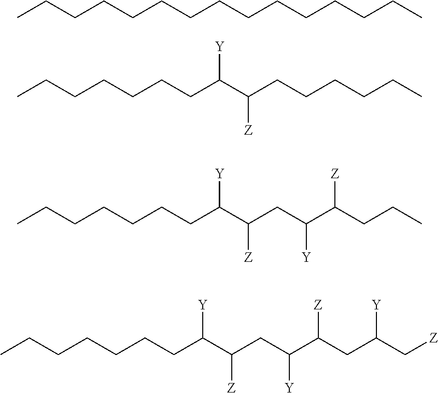
and R_{3,}R₄, m,n,p are as defined in claim 31; 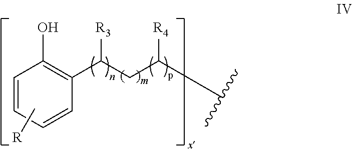
wherein
x is 1,2,3,4,5, or 6;
R is 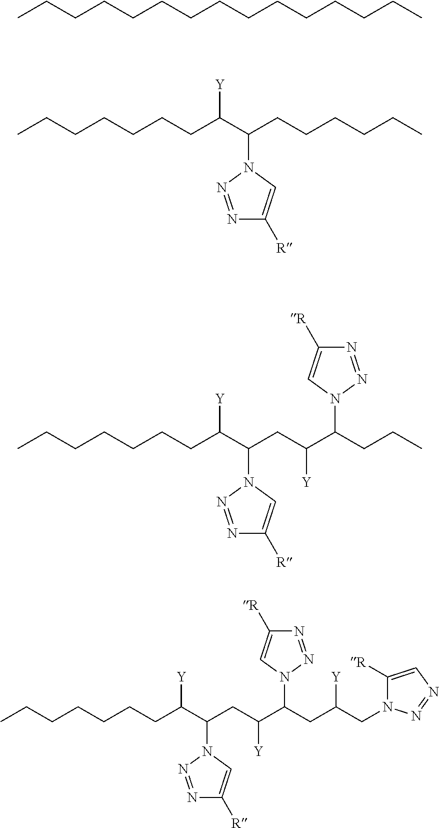
and R_{3,}R₄, m,n,p are as defined in claim 31; 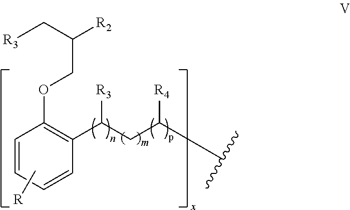
wherein
x is 2, 3, 4, 5, or 6;
R is 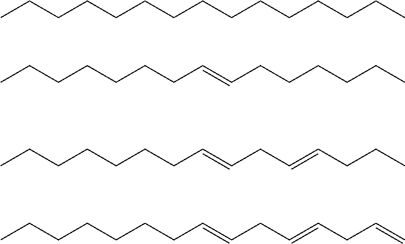
and R_{3,}R₄, m,n,p are as defined in claim 31;
and 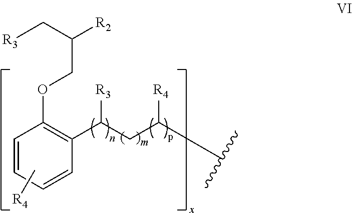
wherein
x is 2, 3, 4, 5, or 6;
R is 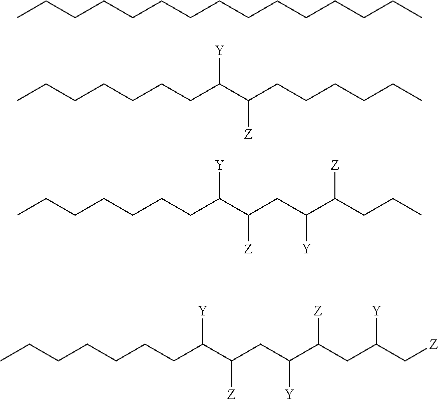
and R₃,R₄,m,n,p are as defined in claim 31.

33. A method for obtaining a cardanol derivative according to claim 31, comprising the steps of:
(a) providing a cardanol selected from saturated cardanol, cardanol monoene, cardanol diene, cardanol triene or a mixture thereof;
(b) condensing of said cardanol or cardanol mixture optionally with an aldehyde or acetal or a second phenol and aldehyde or acetal.

34. The method according to claim 33, wherein before said condensing step said cardanol is freshly distilled, characterized by chromatography, purified and hydrogenated to obtain a saturated cardanol and/or cardanol monoene.

35. The method according to claim 33, wherein said aldehyde is selected from the group consisting of alkylic aldehydes and acrylic aldehydes.

36. The method according to claim 33, wherein said condensation is carried out in the presence of a halogenated solvent and a Lewis catalyst, thereby a polyol of formula I is obtained.

37. The method according to claim 33, wherein said condensation is carried out with paraformaldehyde in the presence of diethanolamine, thereby a polyol of formula II is obtained.

38. The method according to claim 33, wherein the condensation is carried out with a second phenol and an aldehyde or acetal in the presence of an acidic catalyst to obtain a polyol having a polyphenolic structure.

39. The method according to claim 38, wherein said second phenol is a cardanol and/or bears one or more substituents, or is a polyol obtained with the method comprising the steps of
(a) providing a cardanol selected from saturated cardanol, cardanol monoene, cardanol diene, cardanol triene or a mixture thereof;
(b) condensing of said cardanol or cardanol mixture optionally with an aldehyde or acetal or a second phenol and aldehyde or acetal;
wherein said condensation is carried out in the presence of a halogenated solvent and a Lewis catalyst, thereby a polyol of formula I is obtained or wherein said condensation is carried out with paraformaldehyde in the presence of diethanolamine, thereby a polyol of formula II is obtained.

40. The method according to claim 39, wherein the substituents are selected from the group consisting of phenyl, alkyl, alkenyl, aryl, condensed rings, amino, halogen and hydroxy.

41. The method according to claim 38, wherein said acid catalyst is selected from the group consisting of mineral, organic and Lewis acids.

42. The method according to claim 38, further comprising epoxidation of said polyol having a polyphenolic structure with an epoxidizing agent to obtain an epoxidation product containing oxiranic rings, followed by nucleophilic oxiranic ring opening with a nucleophilic agent.

43. The method according to claim 41, wherein said nucleophilic agent is selected from the group consisting of hydrogen, alcohols, ammonia, azides, amines.

44. The method according to claim 42, wherein said epoxidation agent is selected from the group consisting of hydrogen peroxide, epichlorhydrin, peroxyformic acid, peroxyacetic acid, trifluoroperoxyacetic acid, benzyloxyperoxy formic acid, m-chloroperoxybenzoic acid, and any combination thereof.

45. The method according to claim 42, wherein said epoxidizing agent is a peracid, whereby obtaining a polyol of formula III.

46. The method according to claim 45, wherein said nucleophilic agent is an azide and further comprising a 1,3-dipolar cycloaddition reaction with an alkyne in the presence of a suitable catalyst, preferably copper metal or copper sulfate with sodium ascorbate, wherein copper(I) is in the catalytic species, thereby obtaining a polyol of formula IV.

47. The method according to claim 38, further comprising functionalization of phenolic OH groups with epichlorohydrin thereby obtaining a functionalized product containing oxiranic rings, followed by nucleophilic oxixanic ring opening with a nucleophilic agent, whereby obtaining a polyol of formula V.

48. The method according to claim 33, further comprising functionalization of the phenolic OH groups with epichlorohydrin and epoxidation of the product containing oxiranic rings, nucleophilic oxixanic rings opening with an azide and a 1,3-dipolar cycloaddition reaction with an alkyne in the presence of a suitable catalyst, preferably copper metal or copper sulfate with sodium ascorbate, wherein copper is in the catalytic species, thereby obtaining a polyol of formula VI.

49. The method according to claim 46, wherein said alkyne bears substituents and has the triple bond terminal or not.

50. The method according to claim 49, wherein the substituents are selected from the group consisting of acetylene, propyne, phenylacetylene, but-1-yne, but-2-yne.

51. A method for obtaining a polyurethane comprising the steps of:
(a) providing cardanol derivatives according to claim 31;
(b) reacting said cardanol derivatives with isocyanate in the presence of catalysts, if required, and addition of a blowing agent to produce a polyurethane.

52. The method according to claim 51, wherein the isocyanates at least a NCO reacting group selected from the group consisting of 1,4-diisocyanatobutane, 1,6-diisocyanatohexane (HDI), 1,5-diisocyanato-2,2-dimethylpentane, 2,2,4- and 2,4,4-trimethyl-1,6-diisocyanatohexane, 1-isocyanato-3,3,5-trimethyl-5-isocyanatomethylcyclohexane (IPDI), 1-isocyanato-1-methyl-4-(3)-isocyanatomethylcyclohexane, bis-(4-isocyanatocyclohexyl)methane, 1,10-diisocyanatodecane, 1,12-diisocyanatododecane, cyclohexane 1,3- and 1,4-diisocyanate, xylylene diisocyanate isomers, triisocyanatononane, 2,4-diisocyanatotoluene or its mixtures with 2,6-diisocyanatotoluene, diisocyanatodiphenylmethane or technical polyisocyanate mixtures of the diphenylmethane series, or their mixtures thereof.

53. The method according to claim 51, where the amounts of isocyanate and polyphenolic scaffolds are chosen so as to given an NCO:OH equivalent ratio of from 0.5:1 to 2.0:1, preferably from 0.8:1 to 1.5:1.

54. The method according to claim 51, wherein said cardanol derivatives are used in mixture with, other polyols selected from the group consisting of glycerol, sugars, canola oil deriving polyols, soybean oil based polyols, linseed oil based polyols, and castor oil based polyols, in a weight ratio from 95:5 to 5:95.

55. The method according to claim 51, wherein the catalyst is selected from the group consisting of tertiary amines, metal salts, or a mixture thereof.

56. The method according to claim 55, wherein the tertiary amines are selected from the group consisting of triethylamine, pyridine, methylpyridine, benzyldimethylamine, N,N-endoethylenepiperazine, N-methylpiperidine, pentamethyldiethylenetriamine, N,N-dimethylaminocyclohexane, N,N′-dimethylpiperazine.

57. The method according to claim 55, wherein the metal salts are selected from the group consisting of iron(III) chloride, zinc chloride, zinc 2-ethylcaproate, tin(II) octoate, tin(II) ethylcaproate, tin(II) palmitate, dibutyltin(IV) dilaurate, molybdenum glycolate, and/or a mixture thereof.

58. The method according to claim 51, wherein the blowing agents are selected from the group consisting of water, carbon anhydride, fluorocarbons, chlorofluorocarbons, hydrofluorocarbons, perfluorocarbons, and low boiling hydrocarbons.

59. The method according to claim 51, wherein said method further comprise a surfactants addition, said surfactants selected from the group consisting of silicones, fluoro based surfactants, or organic based surfactants.

60. The method according to claim 51, wherein the polyurethanes are added of additives selected from the group consisting of surface-active substances, internal release agents, fillers, dyes, pigments, flame retardants, hydrolysis preventatives, microbicides, levelling assistants, antioxidants, carbon nano-fibers, nano-graphite, expandable graphite, graphite fine powder, graphite oxide, benzoxazines, phyllosilicate, and/or a mixture thereof.
